(19) United States
(12) Patent Application Publication
(10) Pub. No.: US 2022/0054690 A1
(43) Pub. Date: Feb. 24, 2022

(54) Sanitizing steam room

(71) Applicants: Alon Wallach, Ramat Gan (IL); Michael Etgar Eisenstadt, Kfar Saba (IL)

(72) Inventors: Alon Wallach, Ramat Gan (IL); Michael Etgar Eisenstadt, Kfar Saba (IL)

(21) Appl. No.: 16/995,861

(22) Filed: Aug. 18, 2020

(51) Int. Cl. A61L 2/26; A61L 2/00; A61L 2/22; A61H 33/06; A61M 16/16; A61M 16/10; A61K 33/00; A61K 33/20; A61K 9/00

(52) U.S. Cl. CPC A61L 2/26; A61H 2205/02; A61L 2/22; A61H 33/067; A61M 16/16; A61M 16/109; A61K 33/00; A61K 33/20; A61K 9/0073; A61H 33/065; A61L 2202/122; A61L 2202/121; A61L 2202/15; A61L 2202/11; A61L 2202/26; A61M 2205/368; A61M 2205/36; A61M 2205/362; A61M 2210/1039; A61M 2205/3375; A61M 2205/3368; A61M 2205/3633; A61M 2205/584; A61M 2205/587; A61M 2205/3344; A61M 2205/502; A61M 2250/00; A61M 2206/10; A61L 2/0094

(57) ABSTRACT

A steam room for sanitizing people's external body parts and clothes, but also their breathing airways and respiratory tract, to reduce viral load and thereby mitigate the likelihood of spreading or contracting Viral contamination. Steam room has precise temperature and relative humidity control of a heater and mist generator, in which the mist water molecules contain an accurate concentration of a Chlorine or Ethanol based disinfectant, so that the water vapor or combined air/water mixture in the room that comes in contact with the sanitized person reaches but does not surpass the globally permitted concentration of free Chlorine for swimming pools, dressing rooms and spas. The room has thermally isolated quick doors allowing rapid entry and exit, and the signaling to guide the person when to enter, stay and breath, and exit, in the most suitable timing profile for the application.

This is a new patent application without any priority claims.

### FIELD OF THE INVENTION

The present invention is in the field of sanitizing walk thru gates and other sanitizing means for people. More particularly it relates to such sanitizing means for people that comply with regulated health and fitness installations for the General Public, thus allowing the installation and operation of such sanitizing steam room without any special licensing, and making such operation undisputedly not dangerous or controversial.

### DESCRIPTION OF RELATED ART

Sanitizing installation and surfaces that people come in contact with during everyday life is common and done daily for many decades if not centuries, especially in times of pandemics, like the Covid-19 virus outbreak of late 2019. These sanitizing usually take place using sprayers of water with high disinfectants concentration, the most common and cheap being Sodium Hypochlorite, also known as common House Bleach. However, sanitizing the people themselves, in contrast with spraying surfaces and objects, with such active material, is generally forbidden, because disinfectants are poisonous materials.

Since the viruses are usually spread from infected people to other people, it is natural to try and sanitize or clean the people as well.

Many “walk thru” sanitizing gates have popped up in early 2020 in many countries, mainly in places like India, Pakistan, Malaysia, Thailand, Vietnam, etc. Many of those simply “copied” the surfaces spraying into spraying the people from head to toes. The concentration of the active material was naturally decreased 10 or 100 or even 1000 times, so as not to poison people or “bleach” or stain their clothing etc. Additionally some reverted to using “mist” spraying by high pressure sprinklers which generate very small droplets, which reduces the amount of water sprayed over each person, making the walk thru less annoying. This misting however reduced the efficacy of the sanitizing, requiring again higher concentration of active material or looking for less poisonous active materials. A new trend appeared in 2020 in the form of “Hypochlorous Acid” or HOCL which is claimed to be less poisonous and considerably more disinfecting effective than Sodium Hypochlorite but costs many times more while its less poisonous nature is still controversial. It is also less stable after dilution with water and therefore requiring special low dosage pumping into the water tanks of the spraying mechanism. Other options include Ethanol Alcohol based disinfectants that are supposedly less irritating to the skin, eyes and respiration. The efficacy of the misting combined with these new materials still requires concentration levels of 50 PPM (milligram of active material per liter of water) and more to do any effective sanitizing.

As soon as these sanitizing gates came to the market, around the 1^{st} quarter of 2020, the governments, all of them, thru their ministry of health and health experts, started scrutinizing them as illegal means, claiming the gates are making more damage than good, and most of all, missing the main objective of sanitizing the person's source of infecting others, namely his airways and respiratory tract (inner nose and neck), and in some extent also his lungs.

It appears that spraying people with other than pure water will remain banned, and at any rate, it cannot sanitize peoples' airways.

This led to the inescapable question and answer: how do you make people inhale the active material so it does get into the airways, and how do you make it both effective and within the existing regulations ? The answer is; you have to STAY for a period of several tens of seconds (several breathing cycles) in a place with air that includes the active material, and it has to be an environment that is already in use and legal.

Professor of Physics Tsumoru Shintake from ISTGU University in Okinawa Japan published an article on Mar. 13, 2020, titled “Possibility of Disinfection of SARS-CoV-2 (COVID-19) in Human Respiratory Tract by Controlled Ethanol Vapor Inhalation”. He explains the biological effects of inhaling Ethanol alcohol on viruses in very high concentrations of around 40%. His article does not mention any walk thru gates, and he recommends this inhaling only for Medical Crews at first, because Alcohol can do damages to the Tractea (last airway before the lungs).

There have been many nongovernmental suggestions for inhaling diluted disinfectants like house bleach for disinfecting the inside of the human body against Corona virus, but nobody gave a reasonable method to do it in a safe and within known regulations. The present invention tries to give a possible reasoned industrial solution in that direction. With regards to the patentability of this solution, the notion of staying in a hot and humid cabin naturally brings us to the multitude of patents in the Sauna and steam room field. Yes, there are hundreds of such patents in areas covered by class 4/524 and similar classes, regarding Saunas, Steam Rooms and Sweat Rooms.

However, none of those has any mentioning of deliberate mixing of real disinfecting materials or agents into the steam or mist of the room or cabin, in all their different forms and combinations. There probably is a very good reason that nobody tried or thought about it—It's not at all customary or convenient for anybody to be stuck for long minutes in a steamy room full of Chlorine or Alcohol fumes that might end up in fatal Chlorine poisoning or severe damage to the airways and lungs or eyes. The closest patents relate to addition of Therapeutic oils and smells, or alcohol scent for the entertainment in a Sauna, but these of course have no disinfecting or sanitizing effect or negative effect on human health and pose no danger unless somebody is allergic to the administered odor. If someone would really want to introduce a Sauna or Steam Room with Chlorine, the first thing to worry about would have been HOW MUCH Chlorine and how do you make it safe, accurate, non poisonous, and convenient for persons entering a heavily Chlorinated cabin, and for what purpose exactly.

There exists a medical form of administering medicine in the form of mist for helping to improve the lungs function of Asthma patients etc. This includes the medical Nebulizers and even personal inhaler (puffer) like Ventolin. Ventolin has the active material of Salbutamol. In the US it is more known as Albuterol Sulphate. The mist inhaling of this FDA approved medicine is considered as an emergency or quick relief for people having narrowing of their airways. Albuterol must be used with caution and limited doses, and it can have serious side effects. The same medicine is available in pills or injections.

In contrast with the Nebulizer type of medical inhalation, the present invention is not requiring any FDA approval or medical staff, because it does not treat any medical issue, but is rather a preventive measure. Its use resembles “salt breathing rooms” available in various places in the world, either natural inside ancient salt caves and mines, or in spas where big chunks of salt are placed in a room with or without humidity and heating. But Salt is not a recognized poisonous disinfectant like Chlorine, although being the most common substance used by mankind for cleaning (all washing powders), and preparing and preserving food. Salt rooms therefore do not have any dosage issues and their contribution for viral disinfection of airways is close to zero.

### SUMMARY OF THE INVENTION

Addressing the problems in the prior art, and trying to overcome the regulatory limitations and bans around sanitizing people's body and skin, led to the following solution, which can be implemented in several embodiments and ways.

The present disclosure provides a practical sanitizing steam room, or more accurately—warm mist room/cabin, to sanitize the clothes and extremities as well as the airways of people that need to enter into places other than their homes, in which they could be subject to catching or infecting a viral disease like Covid-19 or flue through the interacting and proximity to other individuals. This could be useful for places like praying houses, nursing homes, airports and flights, schools, cinemas and even ordinary workplaces and offices.

The concept is a cabin in which a person gets in contact with a sanitizing substance, but unlike “tunnels” with 2-3 seconds “pass through” between strong nozzles spraying from overhead and sides, with random droplets that can reach undesirable places in the face, eyes etc with high concentration of active materials or quantity (mandatory spraying in every such quick “walk thru” setup for making any worthwhile disinfection), the present invention is not spraying any liquids, but rather is a static misty and warm sauna like room, with quick and easy to enter heat isolated access doors, in which each person enters and stays inside for a nominal time slot of several tens of seconds up to several minutes, a long enough time that on one hand will require the person to inhale the mixed air/humidity/active material, and on the other hand will allow even a super small concentration of active material to create a considerable sanitizing effect that reduces the viral load in the place the viruses multiply—the airways. In essence it's a steam cleaning style/inhalation room for people, with very fine mist, enriched with microscopic doze of sanitizing material.

The invention seeks to show how to make this room into a very precise dosage cleaning cabin, so it qualifies as a benign spa class steam room, with its required “Chlorination” materials administered and tailored in a precise special way so it can be easily proved to any regulatory agency that the concentration of active materials (Free Available Chlorine—FAC) does not exceed the regulations for public or private swimming pools, spas and related dress change rooms.

It must be underlined that this sanitizing room does not claim to heal or cure people from any viral or microbial disease or contamination. It's not a medicine or a medical treatment, and therefore does not require any FDA approvals. Like a swimming pool has its special contribution to human health, so does this sanitizing room, that fully exploits the sanitizing potential of “Chlorination” required by law as general anti bacterial treatment of water in public pools etc.

The numeric examples and calculations of concentrations of active material, temperatures and humidity etc are for helping to understand the principle of the invention, its use and calculations with limitations. The invention is naturally generic and not limited to these numerical examples.

For understanding how this room or cabin is implemented, one must consider the term “humidity”, which is usually referring to “relative humidity”. Relative humidity is the amount of water content present in the air out of the maximum water that can be present in such air before condensation starts—the 100% humidity point. The warmer the air, the more absolute humidity or water quantity that it can contain. For example, 20 degrees Celsius air has a maximum water content of 17 Grams per cubic meter of air, while 40 degrees air has 51 Grams, and 50 degrees has 83 grams. It is evident from this fact that the warmer the air is in the Sauna room, the more water it can contain. For any maximum allowed concentration of active material in the inhaled mixture of air and water, the warmer this mixture is, the more active material that it can legitimately contain. A Wet Sauna with 50 Degrees Celsius air can have exactly 3 times more water and therefore 3 times more absolute quantity of active sanitizing material than a 20 Degrees Celsius air. Each breath of the warmer air will have 3 times more sanitizing power because of the higher water content.

In addition, higher temperatures are known for accelerating biological effects, especially in Chlorination processes. Chlorination is an effect that involves biological “attack” by the Chlorine atoms or ions over envelopes of living cells of bacteria and viruses. This temperature dependence is known as the Q10 rule. It means that each increase of 10 degrees in Celsius temperature, the metabolism and biochemical reaction roughly doubles (Q=2). From 20 C to 40 C it therefore quadruples. So, it can be easily seen that warming the steam room from 20 Celsius to 40 Celsius, has the benefit of increasing quantity of active material by a factor of 3, and by another factor of 4 because of the high temperature metabolism Q10 increase, so a combined increase factor of 12.

Another advantage in using warm mist versus normal water droplets shower of a walk thru tunnel, is that in well warmed room with evenly spread mist reaching high measured relative humidity in the order of 80+ percents, one can consider the mixture of air with its water content as one homogenous wet mixture. Let's take 40 Celsius air with 100% humidity, where the water content is 51 Grams per cubic meter, and the corresponding air weight is 1100 Grams per cubic meter. The total water mass density in this air is therefore only 51/1100 or just 4.6 percent. If the maximum permissible FAC Chlorine concentration in pools that is allowed to come in contact with a person's skin is 3 PPM (Part Per Million, or 0.0001 percent), one might rightly calculate that you can have 22 times more concentration (100/4.6 percents) in the water droplets of the mist, or 66 PPM, that practically have an equivalent or comparable concentration of 3 PPM Chlorine in liquid water, and is therefore complying with the 3 PPM swimming pool regulation, that has no temperature or mist specifications, because it is originally a recognized health limit formed for liquid water.

So, we can see that a uniformly heated and moistened steam room of 40 Degrees Celsius can have up to 22×12=264 more sanitizing affectivity than a randomly spraying tunnel in 20 Celsius, both complying with the 3 PPM regulatory limit.

The price paid for this dramatic increase in sanitizing efficacy is in three aspects: Increased time required for each person to stay in the room, which means a human passage capacity per hour of tens of times smaller than a walk thru spraying tunnel, and the need to have it in the form of a well warmed thermally isolated room with special doors. The heat and humidity loss each time the door is opened plus the time required to stay inside, excludes the newly invented sanitizer from applications that require very high people flow rate, like entrance to Stadiums, theaters etc. The third aspect is energy costs, with the steam room requiring heating and keeping it warm at 40 Celsius, sometimes even 24/7.

The following description of the drawings will explain how it works.

### BRIEF DESCRIPTION OF THE DRAWINGS

The preferred embodiment is illustrated in the following 3 figures.

FIG. 1 is a front view of the sanitizing cabin, with its curtain door and all displays.

FIG. 2 is a side view of the sanitizing cabin, with the ultrasonic humidifier and mixture liquid container.

FIG. 3 is a rear view of the sanitizing cabin.

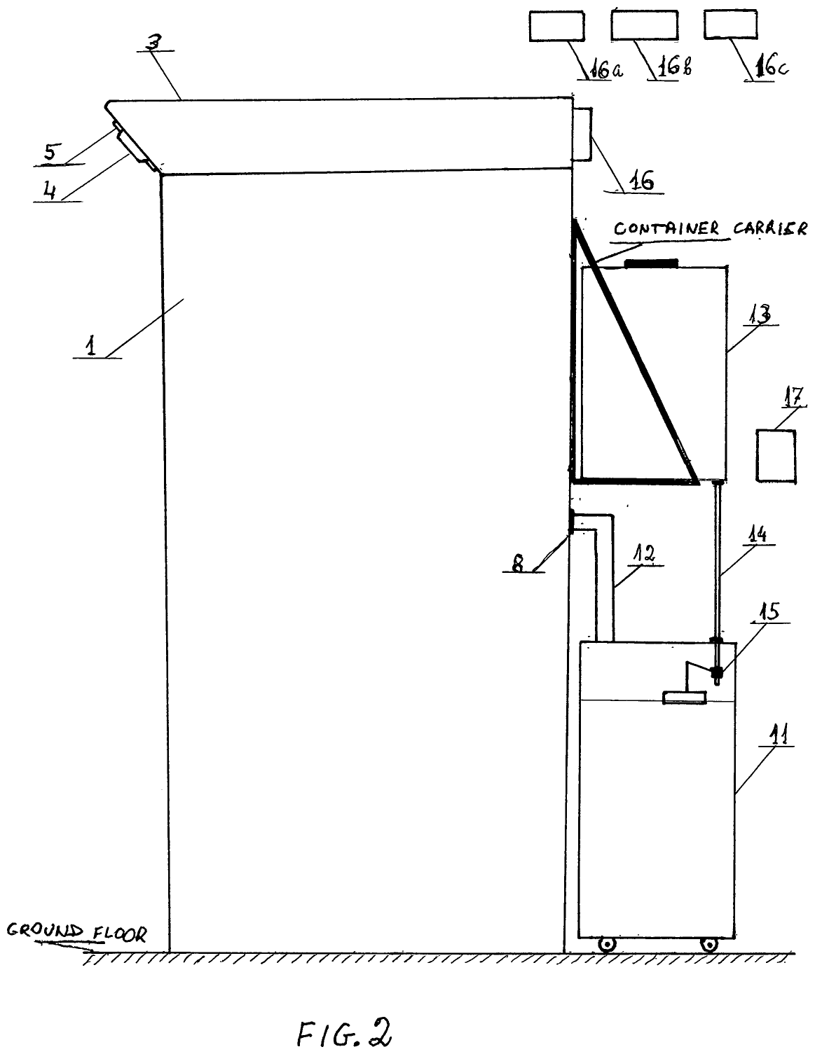

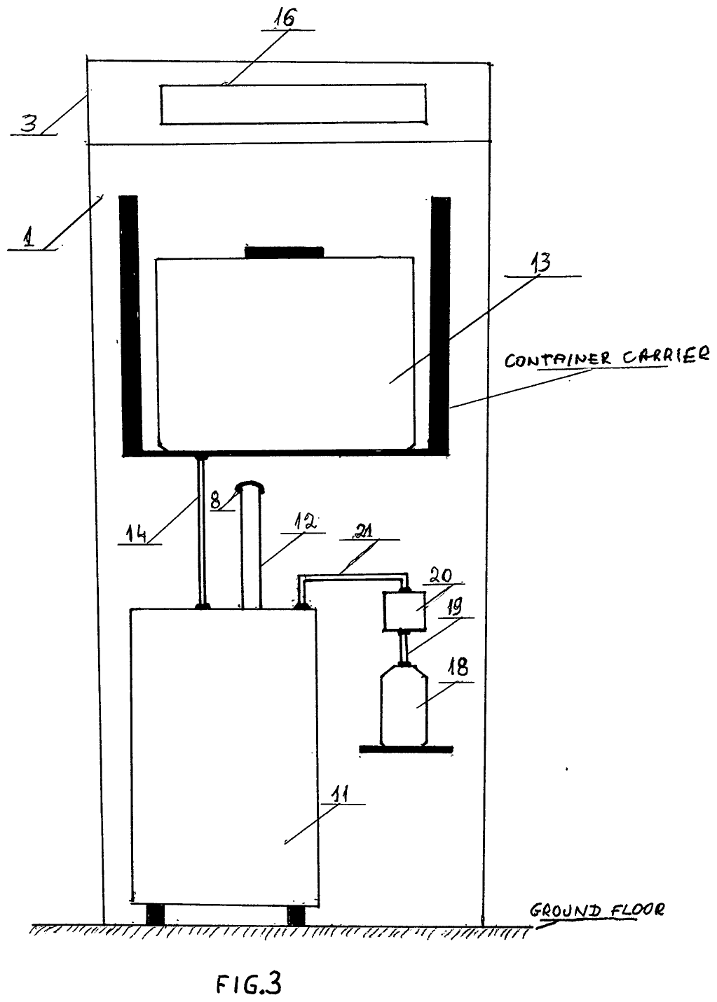

A second and third embodiment will be described using same drawings but with different descriptions and roles of some blocks in FIGS. 2 and 3.

Some blocks have “enlargements” drawings to better explain their looks and roles.

### DETAILED DESCRIPTION

The description is intended mainly to augment the claims, in combination with the drawings. The drawings are merely illustrative block diagrams with an “artist view” of the cabin and its components. It is understood that an actual cabin can look completely different and its blocks located in other positions or locations relative to the cabin's walls and ceiling.

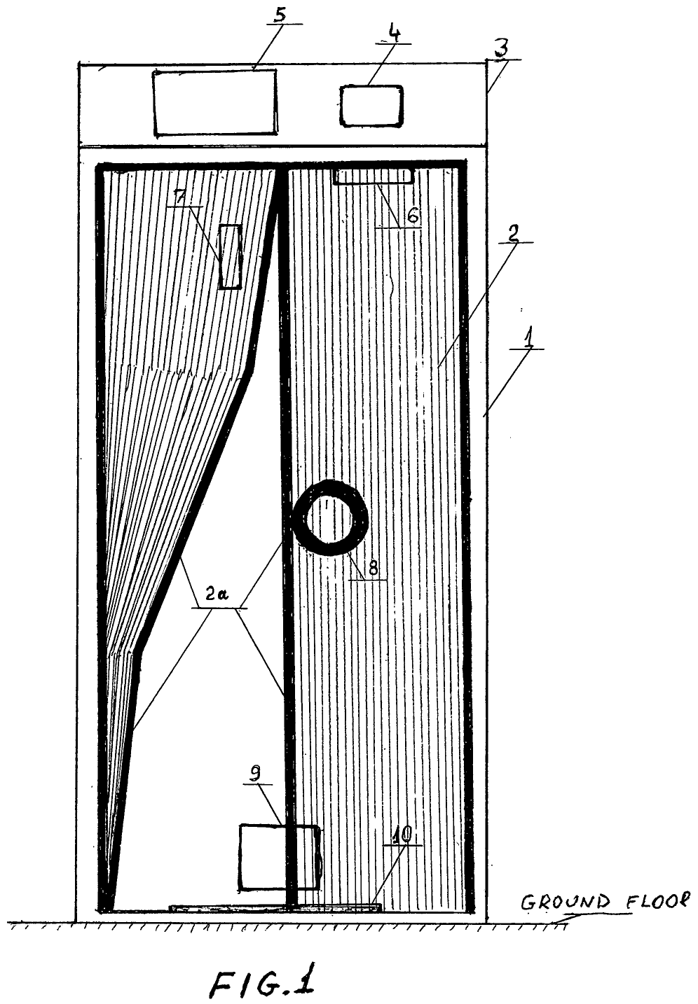

FIG. 1 illustrates a cabin as seen by a person that enters it to be sanitized. The cabin structure having 3 solid walls and a ceiling is defined as “Steam Disinfection Station with insulated panels” numbered “1”. It is a thermally isolated room around 1 m by 1 m and 2 m high. The walls and ceiling can be built from 0.5 mm thick iron sheet inside and out, and a 40 mm thick polystyrene isolation material in between, making it modular to assemble, thermally isolating, and durable. The left, right and ceiling are also appearing in FIGS. 2 and 3. It is obvious that different materials, dimensions and assembly are also possible.

Item number 2 is a two winged thermally insulated curtain (drawn open at its left wing and closed at its right wing). When closed, the two wings join in the center vertical line by magnets 2A. The curtain itself could be made of PVC sheet or equivalent and each wing is secured all along the top and its side of the door, causing it to naturally straighten back and close the gap. The idea is to form a no handle “push in-push out” door that a person just walks through pushing the curtain open, and it opens and closes after him automatically with minimum effort and loss of heat and humidity each time.

For cabins that need to be in the walk thru style, meaning that traffic is only in one direction, an additional curtain door will be located instead of the solid back wall, and all the units connected behind that missing wall will be placed out of the left or right wall.

Above curtain 2 you can see the Electricity Distribution and Control Section 3 that manages the various subsystems. Number 4 is a green/red light indicator that signals to the person when to enter and when to exit the cabin after the sanitizing period has elapsed. Number 5 is a small video screen that plays a loop short video showing how to get in, what to do inside, and when and how to walk out.

Number 6 is a blower that blows downwards to improve the circulation and uniformity of the mist and heating. If it's a door wide horizontal blower located straight above the curtain 2, it can also make an “air curtain” and further reduce heat and humidity losses, or it can even replace the curtain doors.

Number 7 is a temperature and humidity sensor located inside the cabin, roughly at “face height”, not too close to any wall, so it senses correctly the average temperature and humidity of the cabin, at the most important height of the nose and mouth of an average height person.

Number 8 is the cold mist outlet pipe, the edge of the pipe coming from the humidifier 11 placed outside the back wall. It is where the cold mist is pushed into the cabin. This cold mist is made from very small water droplets and is therefore considerably heavier than air, so it will tend to sink towards the floor.

Number 9 is a heat spreader, a heater with blower that draws cold humid air from the lower part of the cabin, heats it by passing it through a hot filament, and pushes it upwards to heat the room, and thereby mix the air evenly in the cabin.

Number 10 is a mat covering the cabin's floor, that gives a good shoe grip and traction even when wet, and it also contains a step sensor that senses a person entering, which can help to synchronize the control and timing system.

FIG. 2 illustrates the same cabin as viewed from the side. Number 1 is the left wall. Numbers 3-5 from FIG. 1 are seen here again but from the side. Number 8 mist outlet pipe is seen going out from Number 11 Ultrasonic humidifier, through a hole in the back wall of Cabin 1, into the cabin. Number 11 is a commercial cold mist ultrasonic humidifier that vibrates a small mass of water adjacent to its ultrasonic transducer vibrating surface, which in turn causes the water molecules to resonant and be thrown up as sub micrometer size droplets. An internal blower inside Humidifier 11 propels those droplets upwards through the pipe and into the cabin.

Ultrasonic humidifiers are unique in not boiling the water like steam generators, but rather atomizing or breaking up the water into very small droplets, not changing their chemical properties or composition. The active material in the broken up water, say Sodium Hypochlorite, will stay in the same concentration in the mist, without any risk of forming super poisonous Chlorine gas. It is there inside the water tank of 11 that the precise concentration of disinfectant has to be added and mixed.

Number 13 is a mixture liquid container which is the main reservoir of water of the sanitizing cabin, that usually lasts for a week of continuous operation. This tank can have about 200 liters of water, already pre mixed at the exact correct concentration (say 66 PPM of above calculation, to give an overall 3 PPM of FAC Chlorine in the inhaled air/mist mixture). For 200 liters of water you will need to add 13.2 cc of Chlorine, and if you use 3% Chlorine bleach, it means 440 cc of Bleach, or roughly two standard cups. Very easy and safe to operate and mix.

Number 13 mixture liquid container is mounted with its bottom side above the height of Ultrasonic humidifier 11 and connected with a Mixture liquid pipe 14. From there the mixture liquid flows by gravitation to Float Control Faucet 15 that keeps the Ultrasonic humidifier tank full by closing the water coming in when the float rises above the preset height, much like a toilette flushing tank.

For shorter operation time per day or when there is a service man available on site on a daily basis, the mixture liquid container is not required, and then the active material will be precisely added into the smaller (25 liters) tank of the Ultrasonic Humidifier 11, or safer, just refilled from ready mixed jars.

The temperature and humidity Control 16B is turning on and off the Heat Spreader 9 and Ultrasonic humidifier 11 respectively, to reach the target temperature and humidity of approx 40 Celsius and 80 percent relative humidity.

Infinite cycle delay timing controller 16C is turning on and off in e.g. 20 seconds Green-Enter, and 40 seconds Red-Wait time slot. The 40 seconds is for enabling the first person to exit, the cabin to disinfect its internal air from whatever viruses the first person left there, before the next person enters. These two times can be adjusted separately according to number of people standing in line for sanitizing etc. For example, in a place where people traffic is low, or medium but evenly spread over the working hours, the Green time can be increased to 40 seconds and beyond, for even better sanitizing, and the Red time to 2 minutes for increased series isolation between consecutive persons entering the cabin.

Electricity Programmable Timer 16A is used to set the activity hours of the Cabin. For example, in a normal 9 to 5 workplace it will be set between 08.00 to 17.30. It turns on the heating and entire system every morning at 08.00 so it will be warm and humid already at 08.30 and turned off after the last employee is out. Such Programmable timers can even make different scheduling for weekend or specific days of the week etc.

Number 17 is a portable Chlorine and PH tester, for making periodical testing of the water that is being misted by Humidifier 11, and its active material concentration, alongside the water present in the bigger water reservoir 13. Number 17 can be an electronic device, or a manual chemically based “color matching”, like the ones used for swimming pool Free Chlorine testing.

The Chlorine or other disinfectant concentration in the humidified liquids is thereby routinely measured by a qualified Chlorination technician, and each measurement and correctional action is recorded in the Sanitizing Cabin diary, similar to public swimming pools.

FIG. 3 illustrates the back (or side, when the cabin has a second magnetic curtain door at the back) of the cabin with its liquids, pipes and related instrumentation. In addition to the parts described above for FIG. 2, FIG. 3 has some additional optional parts that can be seen more clearly when looking from the back side of the cabin, These include parts 18-21. Number 18 is a Pure Disinfectant container with very high concentration active material. Number 19 is Disinfectant Pipe 1, and Number 21 is Disinfectant Pipe 2. Number 20 is a Precision Synchronized Pump that is synchronized and controlled by Timer 16C.

The small sub system comprising parts 18-21 is an active Chlorine injection system that automatically injects a very small dosage of active material into Humidifier 11. This is an alternative way of providing the precise concentration level of disinfectant in the humidified water pushed into the cabin, instead of an already pre mixed water with disinfectant from Mixture Liquid Container 13. Such pumps arrangements like parts 18-21 are used in agriculture and industry but are very difficult to implement when the rate of water quantity flowing and consumed is very low. In the present invention the 200 liters tank 13 lasts for over a week, with average flow rate in the order of 0.5 liter per hour or about 10 cc per minute. These 10 cc are a mixture of several to several tens of ppm of active material. It's impractical to pump such miniscule quantities of active material every minute, and it is more technically feasible to pump a much bigger quantity every hour or so. This means that for example for every 60 red/green light cycles of Timer 16C, Pump 20 will give one portion of disinfectant material of several milligram active material. With such subsystem the cabin can work continuously for very long times (until Container 18 is empty), possibly for months, without the need of Container 13, that will be then replaced by a fresh water feed from a nearby water tap that will be connected to Pipe 14.

Another embodiment of the invention is to replace the separate heating and humidifying with a steam generator or water boiler. This can make the entire sanitizing station cheaper to produce. The steam generator will replace ultrasonic humidifier 11 and heater 9.

In such case the main obstacle would be how to get the active material, namely Chlorine or Alcohol, into the Cabin, and to do it in a very precise way. Chlorine or Sodium Hypochlorite cannot be simply steamed with the water. When you boil water, whatever was added into this water will stay as solid inside the steamer, or escape out as gas, in this case Chlorine gas, a very dangerous poison. What can be done is to add the active material through a Venturi pipe, or with Pump 20, so it “rides” on the rising steam from the steam generator into Pipe 12 and from there to the cabin.

What is claimed is:

1) A sanitizing cabin misted with active disinfectant normally used for disinfecting swimming pools, to sanitize people, their external body parts and clothes, and their airways, comprising:
a. Cabin or room with walls ceiling and at least one door.
b. Reservoirs of water and disinfectant material.
c. Misting said water and said disinfectant and entering them into said cabin in a precise and uniform concentration of active material of said disinfectant.
d. Said cabin has displays that instruct said sanitized person to stay long enough inside said cabin and breath said uniform concentration of active disinfectant, for effectively sanitizing said person's airways.

2) The Cabin as in claim 1 wherein said mist is heated by a heat convector or at least one of the following: Infrared heater, Ceramic heater, heat radiator, heating fan, heating spiral.

3) The Cabin as in claim 1 wherein said cabin comprises walls built from isolated panels with 2 or more thin metal or plastic layers and a thicker layer of insulating air or airy material between said layers.

4) The misted cabin as in claim 1 wherein said misting is done by Ultrasonic Humidifier.

5) The cabin as in claim 2 wherein said heated mist is spread by one or more fans or blowers located in the lower middle or upper parts of said cabin.

6) The cabin as in claim 4, wherein said Ultrasonic Humidifier includes said reservoir of water with said precise concentration of active material, and said humidifier gives mist with same said concentration into said cabin.

7) The cabin as in claim 2 wherein its heating and humidifier are respectively controlled for set temperature and relative humidity inside said cabin, by an electronic on/off controller using a temperature and humidity sensor located inside said cabin.

8) The cabin as in claim 1 wherein at least one of its said doors is a thermally isolating double winged curtain door with magnets on the neighboring edges of each said wings across the vertical line connecting the two wings, to form a push to enter and automatic closing with minimal thermal and humidity loss per opening.

9) The cabin as in claim 1 wherein the disinfectant concentration in said cabin, is marginally below the maximum permitted concentration of said disinfectant for liquid water in swimming pools and spas.

10) The cabin as in claim 1 wherein the said uniform disinfectant concentration of sub claim 1c for the combined mass of water and air mixture in said cabin, is marginally below the maximum permitted concentration of said disinfectant for liquid water in swimming pools and spas.

11) The cabin as in claim 1 wherein said active material disinfectant is Bromine Br or free available Chlorine present in one of the following: Sodium Hypochlorite NaOcl, Hypochlorous acid HOCl, House bleach, Chlorine Dioxide ClO2.

12) The cabin as in claim 1 further comprising a red/green light display and a timer that controls the red and green times, to signal the person to enter on green and exit on red.

13) The cabin as in claim 1 further comprising a mat with pressure step switch that senses when person is inside said cabin, for optimizing the pace of sanitizing and people traffic through said cabin.

14) The cabin as in claim 1 further comprising a video monitor that plays an instructional video or pictures clip that explains to said person how to enter and use said cabin for best sanitizing.

15) The cabin as in claim 1 comprising a boiler of water that creates steam that heats and humidifies said cabin, replacing an ultrasonic humidifier and heater.

16) The cabin as in claim 6 further having a second bigger reservoir in addition to said reservoir of claim 6, which enables nonstop full week operation, and easy mixing of disinfectant.

17) The cabin as in claim 1 further having a pump and high concentration disinfectant tank that together with tap water connection enables months long operation and automatic concentration control of disinfectant.

18) The cabin as in claim 1 wherein it is implemented on a smaller scale as a personal inhalation unit, with a miniature ultrasonic humidifier and its water tank with disinfectant, and possibly also a miniature heat convector, and a hood or mini tent that covers a small volume of air, big enough to allow a person to open the hood and insert his head or torso inside for several minutes or so of sanitizing his airways, with same active material concentrations as the bigger man size walk in cabin of claim 1.

19) The cabin of claim 1 where said sanitized people includes pets
